(19) United States
(12) Patent Application Publication
(10) Pub. No.: US 2013/0289361 A1
(43) Pub. Date: Oct. 31, 2013

(54) FETAL MONITORING TATTOO

(71) Applicants: Lauren Renee Bridge, Waukesha, WI (US); David Momont, Waukesha, WI (US); Brian Dentino, Waukesha, WI (US); Jiaqi Wu, Waukesha, WI (US)

(72) Inventors: Lauren Renee Bridge, Waukesha, WI (US); David Momont, Waukesha, WI (US); Brian Dentino, Waukesha, WI (US); Jiaqi Wu, Waukesha, WI (US)

(73) Assignee: GENERAL ELECTRIC COMPANY, Schenectady, NY (US)

(21) Appl. No.: 13/459,991

(22) Filed: Apr. 30, 2012

(51) Int. Cl. A61B 5/04; A61B 5/0205; A61B 5/0245; A61B 5/11; A61B 5/0478; A61B 5/0408

(52) U.S. Cl. 600/301; 600/382

(57) ABSTRACT

Systems, apparatus, and methods are disclosed for fetal monitoring. An example fetal monitoring device includes a removably adherent connector to connect the device to a wearer and conform the device to a contour of the wearer. The example device includes a sensor including epidermal electronics to detect physical signal(s) from the wearer and/or the wearer's fetus and collect data related to the signal(s). The example device includes a communication interface to wirelessly transmit the collected data to an external processing device. The external processing device is to process the collected data to generate an output indicative of a processing of the collected data according to a guideline or standard. The fetal monitoring device is to adhere to the contour of the wearer so as to not interfere with clothing or movement of the wearer and to form a close bond to receive signals from the wearer and her fetus.

### FIELD OF THE DISCLOSURE

This disclosure relates generally to fetal monitoring, and, more particularly, to a flexible wearable sensor device for fetal monitoring.

### BACKGROUND

During pregnancy, particularly soon before birth, monitoring of a mother and her fetus is important. Lack of monitoring can result in harm to both mother and fetus. Improved monitoring to help fetal assessment can aid in detection of adverse conditions in both the fetus and the mother and hopefully improve outcomes.

### BRIEF SUMMARY

Certain examples provide methods and systems for fetal monitoring.

An example flexible, wearable fetal monitoring device includes a removably adherent connector to connect the device to a wearer and conform the device to a contour of the wearer. The example device includes a sensor including epidermal electronics to detect one or more physical signals from at least one of the wearer and the wearer's fetus and collect data related to the one or more signals. The example device includes a communication interface to wirelessly transmit the collected data to an external processing device. The external processing device is to process the collected data to generate an output indicative of a processing of the collected data according to a guideline or standard. The fetal monitoring device is to adhere to the contour of the wearer so as to not interfere with clothing or movement of the wearer and to form a close bond to receive signals from the wearer and her fetus.

Certain examples provide a tangible computer readable storage medium including instructions for execution by a processor, the instructions when executed implementing a method of fetal monitoring. The example method includes monitoring physical signals from a mother and her fetus using a flexible, wearable fetal monitoring device removably adhering to the mother's abdomen, the fetal monitoring device including epidermal electronics to gather the physical signals. The example method includes wirelessly transmitting the collected data to an external processing device via a communication interface included in the fetal monitoring device. The example method includes processing the collected data using the external processing device to generate an output indicative of a processing of the collected data according to a guideline or standard. The example method includes outputting an indication to the mother based on the processing of the collected data. The fetal monitoring device is to adhere to the contour of the wearer so as to not interfere with clothing or movement of the wearer and to form a close bond to receive signals from the wearer and her fetus.

Certain examples provide a method of fetal monitoring. The example method includes monitoring physical signals from a mother and her fetus using a flexible, wearable fetal monitoring device removably adhering to the mother's abdomen, the fetal monitoring device including epidermal electronics to gather the physical signals. The example method includes wirelessly transmitting the collected data to an external processing device via a communication interface included in the fetal monitoring device. The example method includes processing the collected data using the external processing device to generate an output indicative of a processing of the collected data according to a guideline or standard. The example method includes outputting an indication to the mother based on the processing of the collected data. The fetal monitoring device is to adhere to the contour of the wearer so as to not interfere with clothing or movement of the wearer and to form a close bond to receive signals from the wearer and her fetus.

### BRIEF DESCRIPTION OF THE DRAWINGS

FIG. 1 depicts an example wearable monitor for a mother to monitor fetal and other signal information.

FIG. 2 illustrates an example functional block diagram of a wearable sensor device for fetal monitoring.

FIG. 3 illustrates a block diagram of an example clinical monitoring system.

FIG. 4 depicts a flow diagram for a method of fetal monitoring.

FIG. 5 is a block diagram of an example processor system which may be used to implement systems, apparatus, and methods described herein.

### DETAILED DESCRIPTION

Certain examples provide a flexible, wearable fetal monitoring device (e.g., a fetal monitoring “tattoo”). For example, an expecting mother places a temporary tattoo with circuit elements (e.g., epidermal electronics) on her skin. The embedded circuit detects signals such as an electrocardiogram (EKG), electroencephalogram (EEG), baby motion, and/or other physical signal from the mother and baby and transmits the signal data to an external device such as a smartphone, watch, computer, or other electronic device. The external device isolates the fetal EKG, EEG, and motion signals and displays derived vitals and/or other information, for example. The external device may feature a notification system, for example, to generate an alarm or alert based on the captured data and/or associated analysis. In certain examples, the notification and display of information to the mother may help reduce anxiety as well as an incidence of repeated ultrasounds and/or other images/tests ordered out of fear with respect to the health of the fetus.

In certain examples, a temporary tattoo (e.g., a heart- or other-shaped tattoo) containing epidermal electronics is applied to an expectant mother's skin (e.g., on her abdomen). The tattoo device transmits EKG, EEG, motion, and/or other physical signals from baby (and, optionally, from the mother) to an external processing device for further handling. The external processing device includes hardware and/or software to isolate the fetal signs and display derived vitals, for example. The external processing device may also include a notification system to alert a user (e.g., the mother or the mother's clinician) if vitals are outside of a specified threshold based on hospital and/or other clinical standards, for example. In certain examples, technology such as Bluetooth™, radio frequency identification, near field communication, etc., can be used to send the signals from the tattoo device to the external processing device (e.g., a watch, smartphone, computer, etc.). The mother can view the fetal vitals and be alerted through visual and/or audible cues if the vitals are out of the specified threshold, for example

Certain examples help the mother experience increased peace of mind knowing that she will be alerted if there is a problem with the baby. Having a non-invasive and easy-to-use tattoo is a minimal cost for maintaining peace of mind. The mother can view the vitals on an external device quickly and easily in any setting, for example.

Certain examples provide a noninvasive and lightweight monitoring device, resembling a temporary tattoo that is placed on the mother's skin. The device is customizable as the mother can use different external devices to view the vitals and receive notifications, for example. The monitoring device is flexible such that it adheres to the mother's skin and conforms to the contour of the mother's skin so as not to restrict movement, interfere with clothing, etc.

Certain examples provide a monitoring device using wireless transmissions to send signals to external device(s) for processing, storage, further output, etc. Further, output of the external device(s) can be used to trigger notifications, which can be used to alert the mother or some other third party of unusual and potentially dangerous fetal conditions, or simply reassure any interested parties that the fetus is still healthy, for example.

In certain examples, the monitoring tattoo includes sensors and a communication interface to collect the information and relay or transmit it to another device for processing. In certain examples, tiny curled wires are provided in a flexible membrane to adhere to soft body tissue and gather sensor data. The monitoring tattoo is flexible and removably attachable to the skin (e.g., at or near the woman's abdomen). The monitoring tattoo includes a wireless transmitter to send data to be collected and analyzed to trigger a notification (e.g., an alert, an alarm, a message, a confirmation, a warning, etc.). The monitoring tattoo enables the mother to monitor and receive feedback regarding her baby (and herself) alone or in conjunction with a clinician, for example.

Certain examples may be implemented and/or used in conjunction with an information system for hardware enterprise. For example, a business and application information system may include one or more clinical applications, a processor, data storage, etc. The application may be a clinical application such as a perinatal, radiology, cardiology, and/or other application. Certain examples may provide an architecture and framework for a variety of clinical applications, for example.

An example clinical information system may include a clinical application such as, for example, an advantage workstation (“AW”) and/or Centricity™ product, both manufactured by GENERAL ELECTRIC®. The example clinical information system may also include a radiology information system (“RIS”), a picture archiving and communication system (“PACS”), an electronic medical record (“EMR”), a laboratory information system (“LIS”), a monitoring and/or other perinatal application, an interface, a data store, and one or more workstations, for example. In some embodiments, one or more of the example systems may be implemented remotely via a thin client and/or downloadable software solution. Furthermore, one or more components of the example clinical system may be combined and/or implemented together. Image information, vital sign information, patient history, laboratory test results, etc., can be entered into and processed by one or more of the clinical systems, for example.

Example interface connections may be implemented by, for example, a Wide Area Network (“WAN”) such as a private network or the Internet, a Local Area Network (“LAN”), etc. Accordingly, an example interface may include one or more communication components such as, for example, an Ethernet device, an asynchronous transfer mode (“ATM”) device, an 802.11 device, a DSL modem, a cable modem, a cellular modem, a radio frequency identification (“RFID”) device, a Bluetooth™ communication device, a near field communication (“NFC”) device, etc.

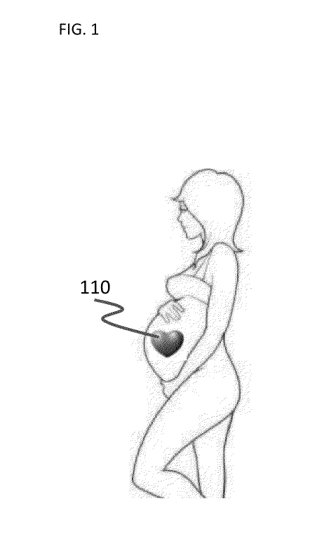

FIG. 1 depicts an example wearable monitor for a mother to monitor fetal and other signal information. The monitor or monitoring device 110 may resemble a tattoo (e.g., a temporary tattoo) or sticker and removably adheres to the woman's skin. The monitor 110 molds to the contour of the woman such that it can receive a quality signal from the fetus and such that it does not interfere with clothing, movement or other mobility of the woman wearing the monitor 110.

In certain examples, the monitor 110 includes circuitry, such as epidermal electronics, that can be placed, rubbed on, and/or otherwise removably or temporarily adhered to the patient. The epidermal electronics patches can detect and record a series of signals to monitor fetal and/or mother health, etc., without limiting the mother to large external machines, belts, vests, and/or other devices with taped or glued electrodes, etc. In certain examples, the electronics and other components in the monitor 110 can stretch and bend with the patient. For example, the circuitry can be fabricated as tiny, squiggled wires, which allows the circuits to bend, twist, scrunch and stretch while maintaining functionality.

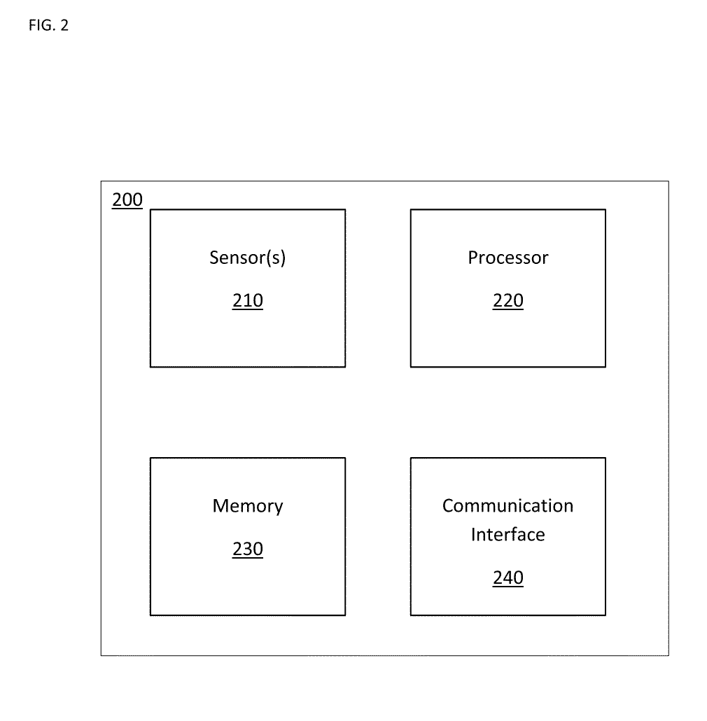

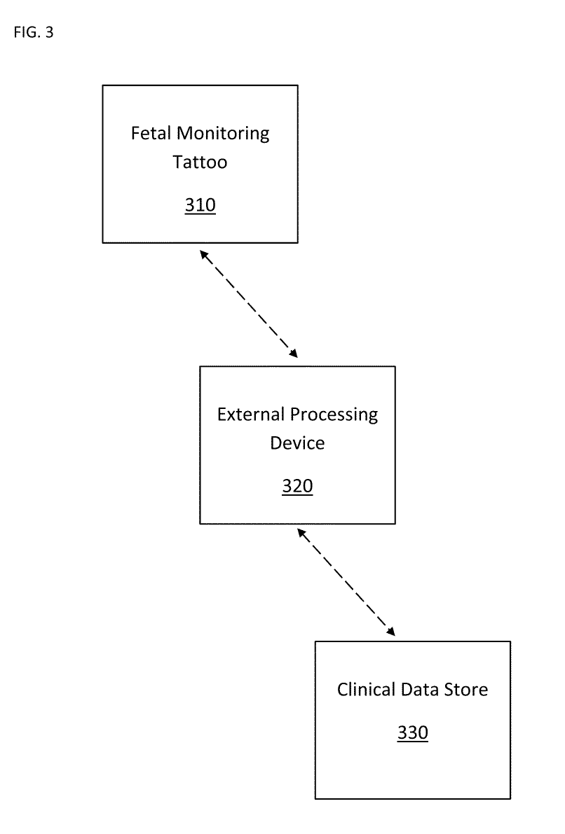

In certain examples, the monitor 110 is encased in water-soluble plastic and can be transferred to the skin just like a temporary tattoo-transfer, with a backing that peels off. The wearer is not bothered by the device 110 because it adheres to the woman's skin based on slight electric forces between molecules (e.g., van der Waals forces). Alternatively, the device 110 can be otherwise removably adherent to the wearer using tiny hooks, removable adhesive, etc. In certain examples, the monitor 110 is protected with a water- and/or other liquid-repellant covering or spray to last for several days. The monitor 110 includes electronics to monitor physiologic signals and a wireless transmitter or other interface to transmit data from the monitor 110 to an external device, as described in more detail in connection with FIGS. 2 and 3 below. In certain examples, the monitor 110 can draw power from induction, miniature solar cells, small battery, etc. The electronics can detect electrical changes beneath the skin and generate signals to be sent through an algorithm to differentiate between different signal indications to drive different alerts, outcomes, indicators, etc.

FIG. 2 illustrates an example functional block diagram of a wearable sensor device for fetal monitoring. The wearable fetal sensor device 200 includes one or more sensors 210, a processor 220, a memory 230, and a communication interface 240, for example.

The sensor(s) 210 collect data from the mother and her fetus and provide the data to the processor 220 and/or memory 230. The processor 220 processes the sensor 210 data and stores the data in the memory 230, for example. The communication interface 240 transmits the data from the device 200 to an external system for further processing, analysis, reporting, monitoring, etc. For example, fetal waveform data can be generated using processed data from the device 200. Fetal and/or mother EKG, EEG, motion, and/or other physical signal data can be captured by the sensor(s) 210, processed by the processor 220 and relayed by the communication interface 240 to an external processor for further processing, for example.

FIG. 3 illustrates a block diagram of an example clinical monitoring system. The example system 300 includes a fetal monitoring tattoo 310, an external processing device 320, and a clinical data store 330.

As discussed above, the fetal monitoring tattoo 310 captures physical signal data from a fetus and/or mother via a flexible monitoring device removably adhering to the mother. The flexible tattoo 310 includes one or more sensors to capture EKG, EEG, heart rate, motion (e.g., contractions, fetal movement, etc.), and/or other signal data, for example. By adhering closely to the contour of the mother, not only does the tattoo 310 avoid interference with clothing and movement but it also receives a strong, reliable signal from the fetus in the mother. The tattoo 310 is able to monitor fetal and/or maternal signals without the need for a mesh, vest, belt, or other multi-node monitoring array, for example.

The external processing device 320 receives the data from the fetal monitoring tattoo 310 and can process the data to monitor status of the mother and fetus. The external processing device 320 can be a smartphone, tablet computer, laptop, notebook computer, desktop computer, and/or other processing device, for example. The external processing device 320 can perform signal processing and data analysis on information received from the tattoo 310, for example. One or more filters can be applied to received signal data, for example. Monitored data can be compared to clinical standards, certain thresholds, patient-specific norms, etc. An alarm, notification, etc., can be triggered based on such a comparison, for example.

In certain examples, the external processing device 320 is configured for the patient to view data, alarms/alerts, set thresholds, record or otherwise memorialize data, etc.

In certain examples, the external processing device 320 can send data to the clinical data store 330 for archiving, further processing, output to a clinician and/or clinical system, etc.

While an example manner of implementing the methods and systems described herein has been illustrated in FIGS. 2-3, one or more of the elements, processes and/or devices illustrated in FIGS. 2-3 may be combined, divided, re-arranged, omitted, eliminated and/or implemented in any other way. Further, the example methods and systems described herein of FIGS. 2-3 may be implemented by hardware, software, firmware and/or any combination of hardware, software and/or firmware. Thus, for example, any of the examples could be implemented by one or more circuit(s), programmable processor(s), application specific integrated circuit(s) (ASIC(s)), programmable logic device(s) (PLD(s)) and/or field programmable logic device(s) (FPLD(s)), etc. When any of the method or system claims of this patent are read to cover a purely software and/or firmware implementation, at least one of the example elements are hereby expressly defined to include a tangible computer readable medium such as a memory, DVD, CD, Blu-ray, etc. storing the software and/or firmware. Further still, the example methods and systems of FIGS. 2-3 may include one or more elements, processes and/or devices in addition to, or instead of, those illustrated in FIGS. 2-3, and/or may include more than one of any or all of the illustrated elements, processes and devices.

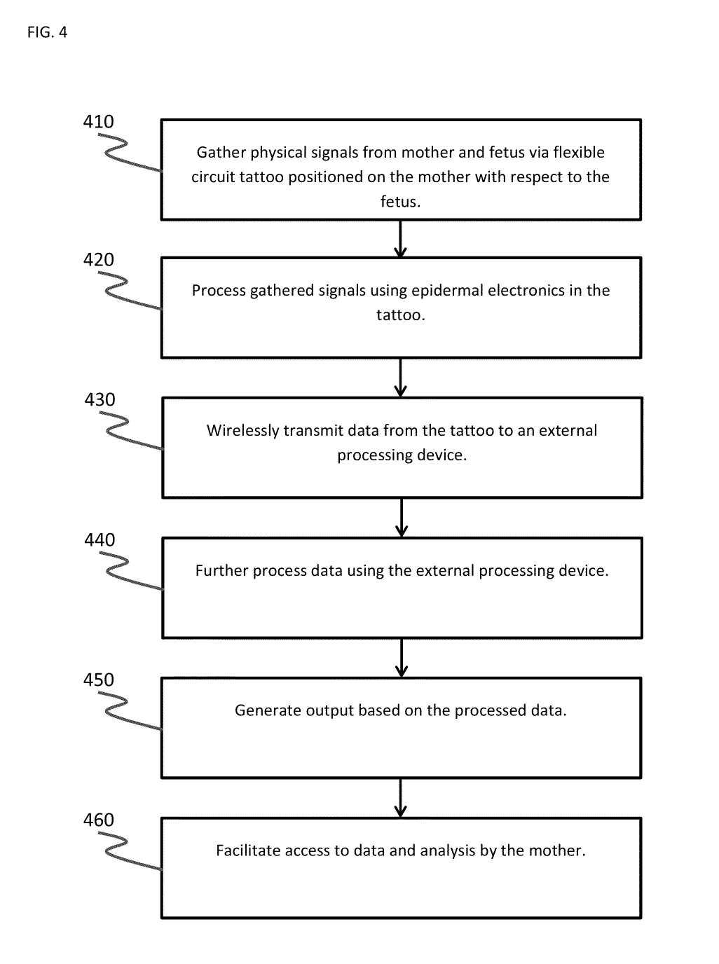

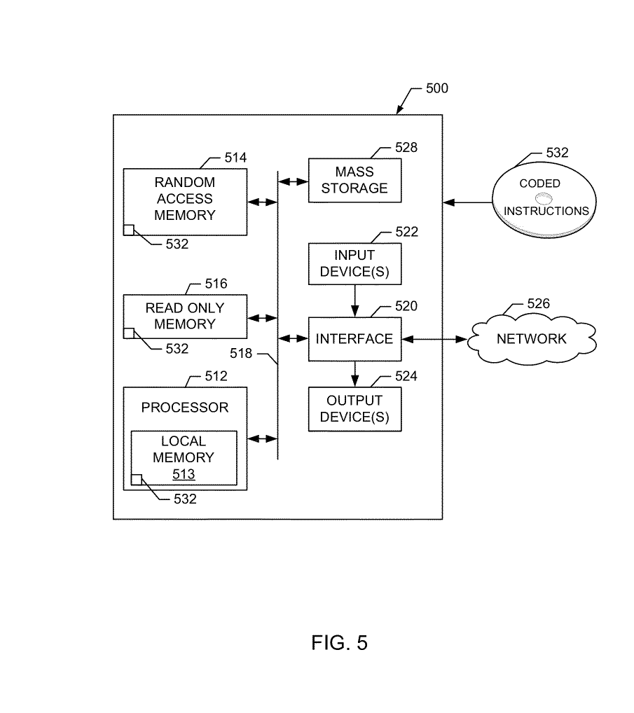

A flowchart representative of example machine readable instructions for implementing the methods and system described herein is shown in FIG. 4. In this example, the machine readable instructions comprise a program for execution by a processor such as the processor 512 shown in the example computer 500 discussed below in connection with FIG. 5. The program may be embodied in software stored on a tangible computer readable medium such as a CD-ROM, a floppy disk, a hard drive, a digital versatile disk (DVD), a Blu-ray disk, or a memory associated with the processor 512, but the entire program and/or parts thereof could alternatively be executed by a device other than the processor 512 and/or embodied in firmware or dedicated hardware. Further, although the example program is described with reference to the flowchart illustrated in FIG. 4, many other methods of implementing the example methods and systems may alternatively be used. For example, the order of execution of the blocks may be changed, and/or some of the blocks described may be changed, eliminated, or combined.

As mentioned above, the example processes of FIG. 4 may be implemented using coded instructions (e.g., computer readable instructions) stored on a tangible computer readable medium such as a hard disk drive, a flash memory, a read-only memory (ROM), a compact disk (CD), a digital versatile disk (DVD), a cache, a random-access memory (RAM) and/or any other storage media in which information is stored for any duration (e.g., for extended time periods, permanently, brief instances, for temporarily buffering, and/or for caching of the information). As used herein, the term tangible computer readable medium is expressly defined to include any type of computer readable storage and to exclude propagating signals. Additionally or alternatively, the example processes of FIG. 4 may be implemented using coded instructions (e.g., computer readable instructions) stored on a non-transitory computer readable medium such as a hard disk drive, a flash memory, a read-only memory, a compact disk, a digital versatile disk, a cache, a random-access memory and/or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals. As used herein, when the phrase “at least” is used as the transition term in a preamble of a claim, it is open-ended in the same manner as the term “comprising” is open ended. Thus, a claim using “at least” as the transition term in its preamble may include elements in addition to those expressly recited in the claim.

FIG. 4 depicts a flow diagram for a method of fetal monitoring. At block 410, physical signals from a mother and her fetus are gathered via a flexible circuit tattoo positioned on the mother with respect to the fetus. At block 420, gathered signals are processed via epidermal electronics included in the tattoo.

At block 430, processed signals are wirelessly transmitted to an external processing device. For example, signals are transmitted from the tattoo to a smartphone (e.g., an iPhone™) including an application or other software to further process the received data.

At block 440, the external processing device processes the received data. For example, one or more filters are applied to the data, one or more thresholds are compared to the data, etc. At block 450, an output is generated based on the data processing. For example, a warning may be triggered for a low fetal heart rate and/or mother's heart rate, a fetal and/or maternal waveform may be shown, an audible beep or series of beeps may sound, etc. Color-coded and/or icon-based alerting can be provided, for example. At block 460, the mother is provided with access to view alerts/alarms/notifications, underlying data (e.g., values and/or waveforms), etc., as well as to log information, request further assistance, route to a clinician, adjust settings, etc. A clinician can be provided with a same or different view of gathered data and analysis, including fetal waveforms and other monitoring and/or historical data regarding the mother, the baby, norms, etc.

FIG. 5 is a block diagram of an example computer 500 capable of executing the instructions of FIG. 4 to implement the methods and system described herein. The computer 500 may be, for example, a server, a personal computer, a mobile phone (e.g., a cell phone), a personal digital assistant (PDA), an Internet appliance, a DVD player, a CD player, a digital video recorder, a Blu-ray player, a gaming console, a personal video recorder, a set top box, or any other type of computing device.

The system 500 of the instant example includes a processor 512. For example, the processor 512 may be implemented by one or more microprocessors or controllers from any desired family or manufacturer.

The processor 512 includes a local memory 513 (e.g., a cache) and is in communication with a main memory including a volatile memory 514 and a non-volatile memory 516 via a bus 518. The volatile memory 514 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 516 may be implemented by flash memory and/or any other desired type of memory device. Access to the main memory 514, 516 is controlled by a memory controller.

The computer 500 also includes an interface circuit 520. The interface circuit 520 may be implemented by any type of interface standard, such as an Ethernet interface, a universal serial bus (USB), and/or a PCI express interface.

One or more input devices 522 are connected to the interface circuit 520. The input device(s) 522 permit a user to enter data and commands into the processor 512. The input device(s) may be implemented by, for example, a keyboard, a mouse, a touchscreen, a track-pad, a trackball, isopoint and/or a voice recognition system.

One or more output devices 524 are also connected to the interface circuit 520. The output devices 524 may be implemented, for example, by display devices (e.g., a liquid crystal display, a cathode ray tube display (CRT), a printer and/or speakers). The interface circuit 520, thus, typically includes a graphics driver card.

The interface circuit 520 also includes a communication device such as a modem or network interface card to facilitate exchange of data with external computers via a network 526 (e.g., an Ethernet connection, a digital subscriber line (DSL), a telephone line, coaxial cable, a cellular telephone system, etc.).

The computer 500 also includes one or more mass storage devices 528 for storing software and data. Examples of such mass storage devices 528 include floppy disk drives, hard drive disks, compact disk drives and digital versatile disk (DVD) drives. The mass storage device 528 may implement a local storage device.

The coded instructions 532 of FIG. 4 may be stored in the mass storage device 528, in the volatile memory 514, in the non-volatile memory 516, and/or on a removable storage medium such as a CD or DVD.

From the foregoing, it will appreciate that the above disclosed methods and systems include establishing a correlation between the emotion of a software application user and the usability and/or intuitiveness of the software application, and allowing for unobtrusively identifying areas for improvement and further software development.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. A flexible, wearable fetal monitoring device comprising:
a removably adherent connector to connect the device to a wearer and conform the device to a contour of the wearer;
a sensor including epidermal electronics to detect one or more physical signals from at least one of the wearer and the wearer's fetus and collect data related to the one or more signals; and
a communication interface to wirelessly transmit the collected data to an external processing device, the external processing device to process the collected data to generate an output indicative of a processing of the collected data according to a guideline or standard,
wherein the fetal monitoring device is to adhere to the contour of the wearer so as to not interfere with clothing or movement of the wearer and to form a close bond to receive signals from the wearer and her fetus.

2. The device of claim 1, wherein the removably adherent connector utilizes molecular forces to removably attach to the wearer.

3. The device of claim 1, wherein the sensor comprises the removably adherent connector formed from a series of hooked wires.

4. The device of claim 1, wherein the sensor collects at least one of electroencephalogram signals, electrocardiogram signals, heart rate signals, and motion signals from at least one of the wearer and the fetus.

5. The device of claim 1, wherein the external processing device comprises a smartphone.

6. The device of claim 1, wherein the external processing device is to provide an interface for the wearer of the fetal monitoring device to receive the output of the processing of the collected data by the external processing device.

7. The device of claim 1, wherein the external processing device is to provide a first output for the wearer and a second output for a clinician.

8. The device of claim 1, wherein the external processing device is to generate an alert based on the processing, the alert to form part of the output.

9. A tangible computer readable storage medium including instructions for execution by a processor, the instructions when executed implementing a method of fetal monitoring, the method comprising:
monitoring physical signals from a mother and her fetus using a flexible, wearable fetal monitoring device removably adhering to the mother's abdomen, the fetal monitoring device including epidermal electronics to gather the physical signals;
wirelessly transmitting the collected data to an external processing device via a communication interface included in the fetal monitoring device;
processing the collected data using the external processing device to generate an output indicative of a processing of the collected data according to a guideline or standard; and
outputting an indication to the mother based on the processing of the collected data,
wherein the fetal monitoring device is to adhere to the contour of the wearer so as to not interfere with clothing or movement of the wearer and to form a close bond to receive signals from the wearer and her fetus.

10. The tangible computer readable storage medium of claim 9, wherein the sensor collects at least one of electroencephalogram signals, electrocardiogram signals, heart rate signals, and motion signals from at least one of the wearer and the fetus.

11. The tangible computer readable storage medium of claim 9, wherein the external processing device comprises a smartphone.

12. The tangible computer readable storage medium of claim 9, further comprising providing an interface for the wearer of the fetal monitoring device to receive the output of the processing of the collected data by the external processing device.

13. The tangible computer readable storage medium of claim 12, further comprising providing a first output for the wearer and a second output for a clinician.

14. The tangible computer readable storage medium of claim 9, further comprising generating an alert based on the processing, the alert to form part of the output.

15. A method of fetal monitoring, the method comprising:
monitoring physical signals from a mother and her fetus using a flexible, wearable fetal monitoring device removably adhering to the mother's abdomen, the fetal monitoring device including epidermal electronics to gather the physical signals;
wirelessly transmitting the collected data to an external processing device via a communication interface included in the fetal monitoring device;
processing the collected data using the external processing device to generate an output indicative of a processing of the collected data according to a guideline or standard; and
outputting an indication to the mother based on the processing of the collected data,
wherein the fetal monitoring device is to adhere to the contour of the wearer so as to not interfere with clothing or movement of the wearer and to form a close bond to receive signals from the wearer and her fetus.

16. The tangible computer readable storage medium of claim 15, wherein the sensor collects at least one of electroencephalogram signals, electrocardiogram signals, heart rate signals, and motion signals from at least one of the wearer and the fetus.

17. The tangible computer readable storage medium of claim 15, wherein the external processing device comprises a smartphone.

18. The tangible computer readable storage medium of claim 15, further comprising providing an interface for the wearer of the fetal monitoring device to receive the output of the processing of the collected data by the external processing device.

19. The tangible computer readable storage medium of claim 18, further comprising providing a first output for the wearer and a second output for a clinician.

20. The tangible computer readable storage medium of claim 15, further comprising generating an alert based on the processing, the alert to form part of the output.
